(19) United States
(12) Patent Application Publication
(10) Pub. No.: US 2005/0281868 A1
(43) Pub. Date: Dec. 22, 2005

(54) Transdermal delivery system for statin combination therapy

(75) Inventor: Edward M. Lane, Weston, CT (US)

Correspondence Address: ROTHWELL, FIGG, ERNST & MANBECK, P.C., 1425 K STREET, N.W., SUITE 800, WASHINGTON, DC, 20005, US

(73) Assignee: Fairfield Clinical Trials, LLC, Bridgeport, CT (US)

(21) Appl. No.: 11/156,744

(22) Filed: Jun. 21, 2005

Related U.S. Application Data
(60) Provisional application No. 60/580,734, filed on Jun. 21, 2004.
(60) Provisional application No. 60/612,828, filed on Sep. 27, 2004.

(51) Int. Cl. A61K 9/70

(52) U.S. Cl. 424/449

(57) ABSTRACT

The present invention relates to transdermal drug delivery systems for the safer delivery of statin drugs to provide lessened danger or severity of side effects.

This application claims the benefit of prior co-pending U.S. Provisional Application Ser. No. 60/580,734, filed Jun. 21, 2004 and U.S. Provisional Application Ser. No. 60/612,828, filed Sep. 27, 2004, both of which are hereby incorporated by reference in their entirety.

### BACKGROUND OF THE INVENTION

1. Technical Field

This invention generally relates to the field of pharmaceutical science. In particular the invention relates to methods and compositions for treating patients in need of statin drug therapy, for example for hyperlipidemia and hypercholesterolemia syndromes, including but not limited to those associated with diabetic conditions. The methods of embodiments of this invention are designed to produce a reduction or elimination of the side effects which commonly occur with statin drugs and to permit treatment of patients who cannot or do not wish to begin or continue statin therapy due to concomitant drug therapies, potential side effects, etc.

2. Description of the Background Art

Dyslipidemia is a term often used to describe a group of syndromes or conditions involving imbalances or extremes in blood lipid levels. Generally, the term encompasses hyperlipidemia and hypercholesterolemia and other lipid disorders, and may also include conditions such as metabolic syndrome, of which hyperlipidemia and/or hypercholesterolemia are hallmarks. Many of these conditions put a patient at greater risk for coronary artery disease, heart attack and stroke. Such cerebrovascular and cardiovascular events are a leading cause of death in the United States and many other countries. Therefore, there is a great need for methods for treatment of lipid disorders, especially those which predispose a patient to cardiovascular problems such as myocardial infarction, anginal conditions, stroke, coronary artery disease, etc.

Statin drugs (competitive inhibitors of 3-hydroxy-3-methyl-glutaryl coenzyme A reductase, the rate-limiting enzyme in cholesterol synthesis) are known in the art and are effective in lowering serum cholesterol and other blood lipids. Such drugs include, for example, Pravastatin (Pravachol™), Lovastatin (Mevacor™), Simvastatin (Zocor™), Rosuvastatin (Crestor™), Mevastatin, Fluvastatin (Leschol™), Eptastatin, Atorvastatin (Lipitor™), Cerivastatin (Baychol™), Pitavastatin and others. Statin drugs also may affect the risk of clot formation and therefore risk of stroke or heart attack. These drugs also can lower LDL and contribute to lowering triglyceride-rich lipoprotein levels. Statin drugs therefore have been of great benefit to persons suffering from elevated lipid and/or cholesterol levels.

Unfortunately, however, some patients in need of treatment for hyperlipidemia and/or hypercholesterolemia conditions cannot use these drugs due to the necessity or desire to also take drugs which are contraindicated for use with one or more statin drug or due to the presence of dangerous or disturbing side effects associated with statin use. Patient compliance for statin drugs is known to be low, especially over the long term, due to various factors. Side effects can include liver transaminase elevations, hepatitis and liver failure (rare), myopathy, rhabdomyolysis and resulting renal failure (rare), proteinuria not related to myopathy and general malaise. The lipid-lowering effects of statin drugs are dose-related, but the associated side effects also are dose-related. For this reason, the more lipid-lowering effect (the higher the dose), the more the likelihood and potential severity of side effects as well. The FDA approved Rosuvastatin (Crestor™) as safe and effective only at doses of 40 mg or less precisely because of its side effects; the higher 80 mg dose produced unacceptable levels and severity of side effects such as myopathy, rhabdomyolysis, proteinuria, hematuria and elevations in serum creatinine levels, occasionally requiring hospitalization for those in clinical trials.

Other mild side effects associated with statin drugs include headache, GI intolerance and fatigue. Most often, the side effects of statin drugs are not severe and abate when the drug is discontinued, however all of the statin drugs are associated with some complaints such as myalgia, weakness, fatigue, mild creatine kinase elevations and the like. Liver enzymes may rise up to 300% in patients receiving statins, necessitating monitoring liver function as an important adjunct to statin therapy. Myalgia is a significant problem for persons receiving statin drugs, either in the short or long term. Even the milder side effects of statins, such as fatigue and gastrointestinal complaints are unpleasant and can result in a patient stopping statin therapy.

Traditionally, treatment of elevated cholesterol involves administration of a statin drug, and if that drug proves ineffective, increasing the dose until either the cholesterol and/or lipid blood levels are reduced to meet the therapeutic goal or until side effects surface. In some patients, however, it is not possible to titrate the dose of the statin drug to a high enough level to achieve the desired effects on cholesterol and lipids without producing at least some of the side effects mentioned above. In some patients, administration of a resin (for example, DEAE-Sephadex and poly(diallylmethylamine) derivatives) or other drug therapy is added to therapy with the statin drug, but many patients also cannot tolerate this therapeutic regimen. This combination regimen also is associated with at least some of the side effects of statin therapy.

Other cholesterol- and lipid-lowering drugs also are known, for example bile acid sequestrants (such as Cholestyramine, Colestipol), fibrate drugs (such as Clofibrate), Nicotinic acid, Probucol, Niacin, P-aminosalicylic acid, cholesterol absorption inhibitors (such as Ezetimibe) and cholesteryl ester transfer protein inhibitors (such as Torcetrapib). Combination drug therapy in lowering of serum cholesterol using some of these drugs with or without a statin drug has been discussed in the literature. Combination therapy generally is known to be of great benefit, in many cases providing an additive effect. Some drug combinations result in a synergistic effect which allows lower doses of each drug in the combination. In theory, the ability to use lower drug doses in turn can reduce the side effects of each drug while maintaining or even increasing effectiveness. Therefore, combination therapy with two or more compounds that have complementary modes of action can be highly effective while lessening side effects. In some cases, the side effects of two compounds cancel each other out due to opposite effects on the same system. Fixed-dose pharmaceutical products, which contain two or more active ingredients in a single dosage form, also reduce the number of prescriptions and thus administrative costs to the manufacturer (for example in packaging costs) and costs to the end-user (in terms of co-pays, for example). Reducing the complexity of a drug regimen also increases patient compliance and thereby also can increase effectiveness. Combination therapy therefore may be of tremendous benefit.

In the case of statin drugs, however, combination therapy regimens for treatment of hypercholesterolemia and/or hyperlipidemia and related or commonly co-occurring conditions including a statin drug have resulted in a greater incidence of rhabdomyolysis, sometimes severe. Drugs and other compounds which are known to increase a risk of statin-associated muscle problems include, for example, Gemfibrozil, Mibefradil, fibrates, Cyclosporine, macrolide and ketolide antibiotics, S-Warfarin, Digoxin, certain antifungal medications, Niacin, HIV protease inhibitors, Nefazodone, Verapamil, Diltiazen, Amiodarone and grapefruit juice. Acute renal failure is thought to occur more frequently with some of the combinations including statin drugs. Therefore, use of combination therapies involving statins must be undertaken with great care. This is particularly so in persons who have had unsatisfactory results with a statin drug previously, in persons who have a genetic or other predisposition (advanced age, renal insufficiency, hypothyroidism) to rhabdomyelitis and in persons with decreased liver function or concomitant drug use that affects liver enzymes. In many patients, it may not be desirable or worth the risk to continue statin use in monotherapy or combination therapy due to the increased likelihood of severe myopathy, fulminant rhabdomyolysis and acute renal failure. In any case, combination therapy involving statin drugs should be attempted only with caution and full information.

Transdermal administration of drugs is known to avoid the first pass metabolism which occurs with oral administration. Other advantages of transdermal administration include the ability to consistently administer the drug at a zero-order (or at least nearly zero-order) rate and the ability to remove the drug rapidly from the user in cases of adverse effects, toxicity or any other undesirable effect. This is not possible with other methods of administration such as oral or intravenous. An additional advantage is increased patient compliance, since usually administration is required less frequently. Transdermal dosage forms can administer the drug for up to seven days, eliminating the need for daily or more frequent administrations.

Treatment of the common problems of elevated blood lipid and cholesterol, diabetes and high blood pressure has made great strides in the past decade due to the availability of new pharmaceutical products. However, because of side effects, many persons are not able to take full advantage of modern therapies for conditions such as hypercholesterolemia, hyperlipidemia and metabolic syndrome. There is a need in the art for treatment methodologies that reduce or eliminate the dangerous side effects of statin drugs and permit their usage by a greater population.

### SUMMARY OF THE INVENTION

Accordingly, the invention, in one embodiment, provides a transdermal pharmaceutical for treatment of lipid disorders in a mammal which comprises a first statin drug and a pharmaceutical composition selected from the group consisting of a second statin drug, a non-statin cholesterol-lowering drug, a non-statin lipid-lowering drug, an antihyperglycemic drug, an antihypertensive drug, a cardiovascular drug and a drug contraindicated for concomitant administration with the first statin drug. The first statin drug may be Pravastatin, Lovastatin, Simvastatin, Rosuvastatin, Mevastatin, Fluvastatin, Eptastatin, Atorvastatin, Cerivastatin, Pitavastatin or any known statin drug. Preferably, the transdermal pharmaceutical contains a second statin drug or may contain a non-statin cholesterol and/or lipid-lowering drug such as Gemfibrozil, Colesevelam, Fenofibrate, Ciprofibrate, Ezetimibe, Torcetrapib or Niacin. In some embodiments, the invention provides a method of treating a patient in need of statin drug therapy but who is concurrently receiving a drug contraindicated for use with the statin drug which comprises administering to the patient a transdermal pharmaceutical composition as described above.

### DETAILED DESCRIPTION OF THE PREFERRED EMBODIMENTS

Without wishing to be bound by theory, it is believed that the dose-related statin side effects are exacerbated by increased plasma levels that can occur in peaks with oral dosing. These peaks are more pronounced in statin drugs which have fairly short half-lives. Statin drugs, generally speaking, have short half-lives (between about 0.5 and 3 hours). Atorvastatin has a half-life of about 14 hours. Because of these characteristics of statin drugs, the plasma levels often spike and produce levels of the drug that are too high, even if temporary, to avoid undesirable side effects. This effect is present whenever an oral dose of the drug is administered. In order to achieve a steady-state drug level high enough to obtain the desired cholesterol-lowering effect, these too-high doses often are an unavoidable consequence when the drug is administered orally.

When provided in oral dosage forms, greater doses of statin drugs result in greater lowering of serum cholesterol levels, but these larger doses also cause higher peak levels of drug and greater likelihood of cardiac, liver and/or muscle side effects. Administration of certain drugs (for example drugs which interfere with a relevant liver metabolic pathway such as a cytochrome P450 or other liver enzyme) concurrently with statin drugs can increase to an even higher level the peak plasma concentration of the statin drug and are known to increase significantly the likelihood or severity of side effects. Such drugs generally are contraindicated for use with the statin drugs. Any drug that can inhibit, competitively antagonize, induce, genetically influence or is metabolized by a liver enzyme that metabolizes a statin drug can be considered contraindicated for use with an oral statin drug. Such drugs are known in the art. Some important examples are listed below. In some cases, this type of combination or concomitant therapy can be life-threatening due to the side effects. Both statin monotherapy and combination drug compositions with statin drugs therefore must be formulated and dosed with care, since some contraindicated drugs are available over-the-counter and may be self-administered by a patient without the knowledge of the treating physician.

A “contraindicated drug,” as the term is used in this specification, is one that is associated with an increases in the severity or incidence of statin-related side effects when co-administered with an orally delivered statin drug, whether due to metabolic influences or for some other known or unknown reason. The art has described such compounds, including for example niacin and fibrate drugs, which are nevertheless co-administered with an oral statin in some patients in spite of the possibility of increased side effects. Persons of skill in the art are aware of drugs that can increase the incidence or severity of statin side effects and therefore can determine which drugs are “contraindicated” as the term is used in this specification. These “contraindicated” drugs can be administered with increased safety when done so according to embodiments of the present invention.

Drugs that inhibit, induce, genetically influence and/or competitively antagonize one or more metabolic pathway involved in station metabolism are known in the art. Many of these drugs are considered contraindicated for use with or contraindicated for concomitant administration with a particular statin drug because their metabolism can change the pharmacokinetics of a co-administered statin drug. Therefore, such drugs that can negatively affect statin side effects may be used in methods, compositions and kits according to embodiments of the present invention. Many of these drugs are either commonly used in the general population (either as over-the-counter or prescription drugs) and/or are used to treat diseases or conditions that commonly co-exist with conditions for which statin drugs are prescribed. In addition, many of these drugs are such that cessation of treatment with them could be dangerous, so a physician is unlikely to halt their administration merely to be able to also administer a statin drug as well.

Examples of drugs falling in the categories discussed above include, but are not limited to, Paclitaxel, Omeprazole, Ibuprofen, Naproxen, Tolbutamide, Glipizide, Celecoxib, Glyburide, S-Warfarin, macrolide and ketolide antibiotics such as Erythromycin and Clarithromycin, HIV antiviral drugs such as Ritonavir and Saquinavir, Biaxin, Chlorpheniramine, calcium channel blockers such as Nifedipine and Verapamil, Hydrocortisone, Progesterone, Fosamax, Buspirone, Propranolol, Phenobarbital, Phenytoin, Cimetidine, Ranitidine, Tegretol, ketolide antifungal drugs, Cisipride, Lopressor, Digoxin, Midazolam, ergot alkaloid drugs, and others. The package inserts for statin drugs list some compounds which are metabolized through the liver and which may affect statin metabolism; these drugs also fall within the term “contraindicated.”

To reduce the side effects of statin drugs in monotherapy or in combination therapy, and to allow administration to a greater proportion of those in need, as well as to increase patient compliance, it would be desirable to reduce the maximum or peak concentration of the drug in blood while still achieving sufficient steady-state concentrations to achieve the desired cholesterol-lowering effect. Since the statin drugs have a short half-lives, relatively large oral doses must be given to achieve sufficient concentrations in the blood over time, or quite frequent small doses must be given. A clinician therefore is forced to choose between increased risk of dangerous side effects by increasing the oral dose and the decrease in patient compliance that often accompanies frequent dosing schedules. Embodiments of the present invention involve transdermal administration of statin drugs in monotherapy or in combination therapy with other compounds which are administered concurrently or sequentially with the statin drug by transdermal or other routes of administration.

One of the greatest advantages of embodiments of the present invention is the ability to achieve a rapid steady-state blood concentration of a statin drug at the desired level without the undesired sudden increases in concentration or high maximum blood concentration (Cmax) which occur with each oral dose. This invention provides, in some embodiments, combination transdermal therapy, where both a statin drug and a second drug are contained in the same transdermal dosage form and combination therapy in which the statin drug is administered transdermally while a second drug is administered by any route, including orally, intramuscularly, intravenously, subcutaneously, nasally, rectally, vaginally, sublingually, transmucosally, intraperitoneally or any route suitable for the drug in question, including transdermally, but using a second dosage form.

Different statin drugs are metabolized by different enzymes, for example liver enzymes, including enzymes of the cytochrome P450 system. Two or more statin drugs which are metabolized (at least predominantly) by different enzymes or metabolic pathways may be combined advantageously according to embodiments of this invention. For example, Atorvastatin, Lovastatin and Simvastatin are metabolized by Cyp3A4, while Fluvastatin is metabolized by Cyp2C9 and Rosuvastatin is metabolized by Cyp2C9 and Cyp2C19. Cerivastatin, which was removed from the market due to difficulties with side effects, is metabolized by Cyp2C9 and Cyp3A4. Combining different statin drugs can result in an additive effect in cholesterol-lowering while avoiding some of the more troubling side effects when the dosage, administration and kinetics are controlled using embodiments of this invention or when the drugs are administered concomitantly with a “contraindicated” drug. Preferably, the statin drugs combined are metabolized by different mechanisms, but this is not required. Therefore, a preferred combination is, for example, Fluvastatin and either Atorvastatin, Lovastatin or Simvastatin. Each statin drug can be given in a lower dose than it would be if used alone as a monotherapy.

Another consideration in choosing a preferred statin drug for any particular patient is the differing ability of each drug to lower low-density lipoprotein or any particular blood lipid component in relation to the severity and type of the patient's lipid disorder. Skilled physicians are aware of the particular lipid effects of each of the statin drugs and consider it routine to weigh the particular needs of any specific patient with respect to needs for lowering or raising subsets of lipid plasma levels. Therefore, any suitable combination of two or more statin drugs which are effective to normalize lipid and cholesterol levels in a particular patient can be used according to embodiments of the invention and would be within the skill of the ordinary practitioner.

Transdermal patches for use with the invention can be of any design known in the art, including specialized patches for iontophoretic delivery or in conjunction with small electric currents (electroporation), ultrasound or microneedle technology to assist delivery across the skin. Suitable patches include reservoir, matrix, multi-layer, drug-in-adhesive, or any type of patch technology known to the art, with or without a rate-limiting membrane to control diffusion of the active ingredient(s). Transdermal patches for a delivery of more than one drug in the same dosage form can be constructed with a single reservoir, matrix or adhesive which contains both drugs, or if biostability or other compatibility problems exist, can be constructed with two separate reservoirs, adhesives or matrices, one for each compound.

Transdermal administration of various pharmaceutical compositions has been described previously. In drug-in-adhesive patches, a drug is dissolved or suspended directly in the adhesive which contacts the skin. Reservoir transdermal systems include a liquid or semi-liquid compartment containing a drug suspension or solution, separated from the skin by a semi-permeable membrane. In matrix transdermal systems, a drug is contained within a solid or semi-solid matrix which contacts the skin of the user and is surrounded at the perimeter by an adhesive. These different transdermal systems are described in, for example, U.S. Pat. Nos. 4,751,087; 5,372,819; 5,405,317; 6,312,715; 6,322,532, the disclosures of which are hereby incorporated by reference. Exemplary suitable transdermal technologies which are compatible with the present invention include those used in D-TRANS™, E-TRANS™, MICROFLUX™, LATITUDE™, LATITUDE™ DUO, CLIMARA PRO™, and any other transdermal delivery systems known in the art.

Combination or concomitant therapy involving a transdermally administered statin drug can minimize the potential side effects associated with drug-drug interactions by reducing the peak plasma concentration of the statin drug, whether the combination treatment is administered in one dosage form or more than one. Combination therapy or administration in combination generally refers to administration of two or more pharmaceutical active compounds in a single dosage form. Concomitant administration or therapy refers to administration of two or more pharmaceutical active compounds at the same time or in such close proximity in time such that therapeutic levels of each compound exist in the patient at the same or overlapping times. Therefore, the term concomitant administration encompasses combined or combination administration/therapy.

Exemplary drugs which may be administered advantageously in combination with or concomitantly with a transdermally administered statin drug include, for example, a second statin drug, antihyperglycemic drugs (such as Metformin, Glyburide, etc.), antihypertensive drugs (such as Lisinopril, Propranolol, Nifedipine, etc.), fibrate drugs, a cardiovascular drug (such as Nifedipine or Verapamil, etc.), coenzyme Q10 and others. Particularly preferred combinations include a statin drug and a second cholesterol- and/or lipid-lowering drug such as a second statin drug, a fibrate drug, Niacin, a bile acid sequestrant, a cholesterol absorption inhibitor and the like. Other preferred combinations include drugs which are commonly co-prescribed or co-administered with a statin drug and any drug which is contraindicated for use with the statin drug as discussed above.

Some drugs which may affect the metabolism of statin drugs by Cyp3A4 include Amlodipine (Norvasc™), Astemizole (Hismanal™), Clindamycin (Cleocin™), Cyclosporine (Sandimmune™), Erythromycin, Indinavir (Crixivan™), Nelfinavir (Viracept™), Verapamil (Calan™), Ritonavir (Norvir™), Saqinavir (Invirase™), Amiodarone (Cordarone™), Cimetidine (Tagamet™), Clarithromycin (Biaxin™), Diltiazem (Cardizem™), Fluconazole (Diflucan™), Itraconazole (Sporanox™), Ketoconazole (Nizoral™), Mibefradil (Posicor™) and Mefazodone (Serzone™). Any of these drugs should be co-administered with oral statin drugs only with care because of possible metabolic interaction and therefore possible undesired side effects and therefore are considered “contraindicated.” However, when the statin drug or both drugs are delivered transdermally, the risk is lessened and the drugs may be co-administered with a greater degree of safety in accordance with this invention. Compounds that can affect metabolism by Cyp2C9 (which also metabolizes Fluvastatin, Rosuvastatin and Cerivastatin) include, for example, nonsteroidal anti-inflammatory drugs such as Ibuprofen, Phenytoin (Dilantin™), S-warfarin (Coumadin™), Ritonavir (Norvir™), Fluconazole (Diflucan™), Itraconazole (Sporanox™), Ketoconazole (Nizoral™) and Metronidazole (Flagyl™). These compounds may be administered more safely with statin drugs metabolized by Cyp2C9 when following this invention.

The invention therefore is particularly useful for patients who would benefit from statin therapy, but who also are receiving or would otherwise receive therapy with any of the drugs listed above or any drug known to be metabolized by liver enzymes which also metabolize a statin drug. In addition, statin drugs also advantageously are administered together with another drug which is (1) part of a combined strategy to achieve a beneficial lipid and cholesterol profile in a patient when a statin drug alone does not provide complete results or (2) part of a combined strategy to treat a condition often occurring with hypercholesterolemia such as diabetes, metabolic syndrome, hypertension, obesity, cardiovascular disease and the like.

Niacin inhibits free fatty acid mobilization from adipose tissue, which results in reduced hepatic synthesis of VLDL (very low density lipoprotein) and triglycerides. Niacin has beneficial effects on the complete lipid profile. Use of niacin to improve lipid metabolism can be limited, however by intolerance of flushing, the predominant side effect. Sustained release Niacin, though exhibiting decreased flushing, has been associated with increases in adverse hepatic effects and hepatic symptoms (for example fatigue, nausea and anorexia), sometimes severe, which were not seen with immediate release forms of Niacin. Many of these problems appear to have been solved with an intermediate release formulation, taken at bedtime, however it is clear that appropriate dosage and blood levels of Niacin are required to avoid its potential side effects. Therefore, embodiments of this invention provide a formulation for a combination treatment of a statin drug for transdermal administration and niacin, either in a single dosage form or in two dosage forms. Commonly, when administered orally, Niacin is given at about 2 g qd. Blood levels of Niacin may be about 0.1 ng/mL to about 1000 ng/mL, preferably are about 5 ng/mL to about 250 ng/mL and most preferably are about 9.8 ng/mL to about 192 ng/mL in this combination treatment. Those of skill in the art can readily formulate a suitable dosage form to provide this blood level or use formulations already known in the art.

Fibrates decrease triglyceride levels dramatically, and are relatively free of side effects when used alone. Fibrates can compete for binding to proteins, however, so patients undergoing combination therapy with highly protein-bound drugs such as statins must be monitored carefully. Some severe incidences of rhabdomyelitis have occurred in patients receiving combinations of a statin with, for example, Gemfibrozil, by oral administration. Embodiments of this invention provide a combination of a statin drug for transdermal administration with Gemfibrozil or any other known fibrate drug. When provided for administration transdermally, the unpleasant and potentially dangerous side effects of this combination are reduced. A preferred combination of this type includes a statin drug with a fibrate drug such as Gemfibrozil or Fenofibrate. Preferred statin drugs for use in this combination are Atorvastatin, Pravastatin, Simvastatin and Fluvastatin. This combination may be administered with a statin transdermal patch and another dosage form containing the fibrate drug (for example oral or transdermal or any dosage form known in the art) or may be administered in a single transdermal patch containing both a statin and a fibrate drug. In this embodiment of the invention, the blood levels of the fibrate drug preferably are about 5 nM to about 100 nM and most preferably about 10 nM to about 20 nM.

Bile acid sequestrants are agents that bind bile acids in the intestine, inhibiting their re-uptake and cutting off their enterohepatic circulation, thereby increasing conversion of cholesterol to bile acids, which can be excreted. Up-regulation of LDL receptors in the liver also results. These agents have a tendency to at least slightly increase triglyceride levels and generally have not been useful in patients with high or borderline high triglycerides for this reason. These agents, because of their mechanism of action (in the intestine) preferably are not administered to the skin, but are useful according to embodiments of the invention when administered orally in combination with a transdermally administered statin drug. Therefore, this invention also includes embodiments for a combination treatment of a statin drug and a bile acid sequestrant. Preferred combinations in this embodiment of the invention include any statin drug and involve traditional dosing of bile acid sequestrants as is known in the art in combination with a transdermal statin drug. A particularly preferred combination in this embodiment is Atorvastatin and Orlistat (Xenical™).

Specific cholesterol absorption inhibitors, such as Ezetimibe, selectively inhibit uptake of cholesterol from the intestinal lumen. These compounds appear to have the advantage over bile acid sequestrants in that they do not tend to increase triglyceride levels. Therefore, this invention, in one embodiment, includes a combination therapy of a transdermal statin drug and an oral or transdermal cholesterol absorption inhibitor. Transdermal systems containing both a statin drug and a cholesterol absorption inhibitor drug in one dosage form are one embodiment of this invention. Any of the known statin drugs are suitable for combinations of this type, including, for example, Atorvastatin, Fluvastatin and Rosuvastatin and involve traditional dosing of the cholesterol absorption inhibitor as is known in the art. Oral dosages for Ezetimibe commonly are about 5 mg qd to about 20 mg qd and preferably about 10 mg qd.

Doses of statin drugs according to the invention preferably are administered transdermally to achieve plasma concentrations of drug at or near the level which is preferred when administering the compound orally or a dose titrated to achieve the desired lipid-lowering effect as is considered routine by physicians treating patients with statin drugs. In general, starting doses for human administration are about 10-20 mg orally or about 0.1-0.25 mg/kg statin drug. Suitable plasma levels of statin drug are known to those of skill in the art since the compounds per se are known and have been available to the public for several years. Suitable amounts of the related statin drugs are known in the art and can be determined for the transdermal administration as a matter of routine since the drugs are per se known and have been used clinically. Commonly used oral doses of statin drugs include Rosuvastatin: about 5-40 mg per day and preferably about 5-10 mg per day or an amount sufficient to achieve lipid level goals; Simvastatin: about 5-80 mg per day and preferably about 10-40 mg per day or an amount sufficient to achieve lipid level goals; Pravastatin: about 10-40 mg per day and preferably about 10-20 mg per day or an amount sufficient to achieve lipid level goals; Atorvastatin: about 10-80 mg per day and preferably about 10-20 mg per day or an amount sufficient to achieve lipid level goals.

Persons of skill therefore can easily determine a safe and effective dose of these drugs when administering them transdermally according to the invention, in all its embodiments. For example, Pravastatin or other statin drugs can be administered to achieve an AUC of about 1-1000 μg-h/l, and preferably about 100 μg-h/l. Desirable steady state concentrations of the statin drugs in embodiments of this invention may range from about 0.5 ng/mL to about 150 ng/mL and generally range from about 1 ng/mL to about 100 ng/mL. The most preferable ranges are about 10 ng/mL to about 20 ng/mL. It may be possible to use higher steady-state plasma levels than previously were considered safe when using this invention.

The invention described above is further exemplified by the non-limiting examples below.

### EXAMPLES

### Example 1

### Exemplary Pharmaceutical Statin Compositions for Transdermal Administration

Any statin drug known to the art is administered transdermally according to the invention, including, for example, Pravastatin (Pravachol™), Lovastatin (Mevacor™), Simvastatin (Zocor™), Rosuvastatin (Crestor™), Mevastatin, Fluvastatin (Leschol™), Eptastatin, Atorvastatin (Lipitor™), Cerivastatin (Baychol™), Pitavastatin and others. These statin drugs are administered in any combination, together in one transdermal dosage form or in two or more separate transdermal dosage forms. Preferred combinations for administration in a single or in two transdermal delivery systems are:
1. Atorvastatin+Fluvastatin
2. Atorvastatin+Rosuvastatin
3. Lovastatin+Fluvastatin
4. Lovastatin+Rosuvastatin
5. Simvastatin+Fluvastatin
6. Simvastatin+Rosuvastatin

### Example 2

### Exemplary Pharmaceutical Combinations for Transdermal Administration Including a Statin Drug

The following drugs advantageously may be incorporated into a single transdermal product, either in the same compartment or reservoir, or in different and separate compartments or reservoirs if necessary or desired. Any of the combinations below also may be administered in separate dosage forms with the additional drug administered transdermally or by a non-transdermal route. A statin drug, for example Pravastatin (Pravachol™), Lovastatin (Mevacor™), Simvastatin (Zocor™), Rosuvastatin (Crestor™), Mevastatin, Fluvastatin (Leschol™), Eptastatin, Atorvastatin (Lipitor™), Cerivastatin (Baychol™), Pitavastatin or any known statin drug may be administered transdermally, concomitantly with any of the following drugs and classes of drugs:
1. a fibrate drug such as Gemfibrozil, Colesevelam, Fenofibrate or Ciprofibrate.
2. A bile acid sequestrant such as Orlistat.
3. a cholesterol absorption inhibitor such as Ezetimibe.
4. a cholesteryl ester transfer protein inhibitor such as Torcetrapib.
5. Niacin.
6. Coenzyme Q10.
7. Amlodipine.
8. an antihypertensive drug such as an ACE inhibitor (e.g. Lisinopril), an alpha blocker (e.g. Phenoxybenzamine), a beta blocker (e.g. Propranolol) or an angiotensin receptor blocker (e.g. Valsartan), each with or without folic acid.
9. one or more antihyperglycemic such as (1) sulfonylureas (e.g. Chlorpropamide (Diabinese™), Glipizide (Glucotrol™), Glyburide (Micronase™, Glynase™, Diaβeta™), Glimepiride (Amaryl™)); (2) meglitinides (e.g. Repaglinide (Prandin™), nateglinide (Starlix™)); (3) biguanides (e.g. Metformin (Glucophage™)); (4) thiazolidinediones (e.g. Rosiglitazone (Avandia™), Troglitazone (Rezulin™), pioglitazone (Actos™)); and (5) alpha-glucosidase inhibitors (e.g. Acarbose (Precose™), Meglitol (Glyset™))
10. an ACE inhibitor (e.g. Lisinopril) and an antihyperglycemic (e.g. Metformin).
11. an ACE inhibitor (e.g. Lisinopril) and Aspirin.
12. an ACE inhibitor (e.g. Lisinopril), a beta blocker (e.g. Propranolol) and aspirin.
13. Rimonabant (Accomplia™).
14. a cardiovascular drug such as Nifedipine (Procardia™), Verapamil (Calan™) or Clopidogrel (Plavix™).
15. any drug which is metabolized predominantly by the same liver enzyme as the statin drug used, for example Astemizole (Hismanal™), Clindamycin (Cleocin™), Cyclosporine (Sandimmune™), Erythromycin, Indinavir (Crixivan™), Nelfinavir (Viracept™), Verapamil (Calan™), Ritonavir (Norvir™), Saqinavir (Invirase™), Amiodarone (Cordarone™), Cimetidine (Tagamet™), Clarithromycin (Biaxin™), Diltiazem (Cardizem™), Fluconazole (Diflucan™), Itraconazole (Sporanox™), Ketoconazole (Nizoral™), Mibefradil (Posicor™), Mefazodone (Serzone™), nonsteroidal anti-inflammatory drugs, Phenytoin (Dilantin™), S-warfarin (Coumadin™), Ritonavir (Norvir™), Fluconazole (Diflucan™), Itraconazole (Sporanox™), Ketoconazole (Nizoral™) and Metronidazole (Flagyl™), Azithromycin (Zithromax™) and Telithromycin (Ketek™).

1. A transdermal pharmaceutical composition for treatment of lipid disorders in a mammal which comprises
a) a statin drug and
b) a drug contraindicated for concomitant administration with said statin drug.

2. The transdermal pharmaceutical composition of claim 1 wherein said statin drug is selected from the group consisting of Pravastatin, Lovastatin, Simvastatin, Rosuvastatin, Mevastatin, Fluvastatin, Eptastatin, Atorvastatin, Cerivastatin and Pitavastatin.

3. The transdermal pharmaceutical composition of claim 1 wherein said contraindicated drug is a second statin drug.

4. The transdermal pharmaceutical composition of claim 1 wherein said contraindicated drug is a non-statin cholesterol- and/or lipid-lowering drug selected from the group consisting of Gemfibrozil, Colesevelam, Fenofibrate, Ciprofibrate, Ezetimibe, Torcetrapib and Niacin.

5. The transdermal pharmaceutical composition of claim 1 wherein said contraindicated drug is a drug metabolized by a cytochrome P450 enzyme that metabolizes said first statin drug.

6. The method of treating a patient in need of statin drug therapy but who is concurrently receiving a drug contraindicated for use with said statin drug, which comprises administering to said patient a transdermal pharmaceutical composition of claim 1.
